(19) United States
(12) Patent Application Publication
(10) Pub. No.: US 2007/0134244 A1
(43) Pub. Date: Jun. 14, 2007

(54) Combination treatment for pathologic ocular angiogenesis

(75) Inventors: Jason Slakter, Great Neck, NY (US); Karl Csaky, Kensington, MA (US); Patricia Zilliox, Arlington, TX (US)

Correspondence Address: ALCON, IP LEGAL, TB4-8, 6201 SOUTH FREEWAY, FORT WORTH, TX, 76134, US

(73) Assignee: Alcon, Inc., Hunenberg (CH)

(21) Appl. No.: 11/581,500

(22) Filed: Oct. 16, 2006

Related U.S. Application Data
(60) Provisional application No. 60/726,765, filed on Oct. 14, 2005.

(51) Int. Cl. A61K 39/395; A61K 31/573

(52) U.S. Cl. 424/145.1; 514/171

(57) ABSTRACT

The present invention provides a combination therapy for the treatment of pathologic ocular disorders, such as age-related macular degeneration and choroidal neovascularization. The combination therapy of the invention includes administration of anecortave acetate and bevacizumab or ranibizumab.

### BACKGROUND OF THE INVENTION

This application claims priority to U.S. provisional application Ser. No. 60/726,765 filed Oct. 14, 2005.

### 1. FIELD OF THE INVENTION

The present invention relates to the field of treatment of pathologic ocular disorders caused by angiogenesis. More particularly, the present invention provides a combination treatment for patients suffering from such disorders.

### 2. DESCRIPTION OF THE RELATED ART

Pathologic ocular angiogenesis, which includes posterior segment neovascularization, occurs as a cascade of events that progress from an initiating stimulus to the formation of abnormal new capillaries. The inciting cause in both exudative macular degeneration and proliferative diabetic retinopathy is still unknown, however, the elaboration of various proangiogenic growth factors appears to be a common stimulus. Soluble growth factors, such as vascular endothelial growth factor (VEGF), basic fibroblast growth factor (bFGF or FGF-2), insulin-like growth factor 1 (IGF-1), etc., have been found in tissues and fluids removed from patients with pathologic ocular angiogenesis. Following initiation of the angiogenic cascade, the capillary basement membrane and extracellular matrix are degraded and capillary endothelial cell proliferation and migration occur. Endothelial sprouts anastomose to form tubes with subsequent patent lumen formation. The new capillaries commonly have increased vascular permeability or leakiness due to immature barrier function, which can lead to tissue edema. Differentiation into a mature capillary is indicated by the presence of a continuous basement membrane and normal endothelial junctions between other endothelial cells and pericytes; however, this differentiation process is often impaired during pathologic conditions.

Age-related macular degeneration (AMD) is the leading cause of vision loss in persons over the age of 50 (Bressler 1988). The severe vision loss associated with the exudative form of AMD is caused by the growth of abnormal new blood vessels from the choriocapillaris, a process call choroidal neovascularization (CNV). The new vessels tend to bleed, exude serum and promote excessive reparative responses within the macula. These changes, in turn, alter the anatomical relationship between the overlying neurosensory retina and the underlying retinal pigment epithelium (RPE) layer, causing detachment, dysfunction and degeneration of the photoreceptors. In the most severe cases, participants lose the ability to read or perform activities of daily living without aid.

Although the exudative form of AMD is present in only 15-20% of the AMD population, exudative AMD accounts for much of the significant vision loss (Seddon 2001). The clinical course of neovascular AMD is poor. For example, in the subfoveal arm of the Macular Photocoagulation Study (MPS), the untreated natural history group provided some insight into how poor the prognosis is for these participants; 83% of participants lost 2 or more lines of vision at 24 months (Macular Photocoagulation Study Group, 1991). Until recently, the only approved treatment for CNV associated with exudative AMD was laser photocoagulation. Recently, several clinical trials evaluating photodynamic therapy (PDT) with verteporfin for the treatment of AMD participants with subfoveal CNV or intravitreal injection of an anti-VEGF therapy have demonstrated a treatment benefit for many participants. Unfortunately, this benefit was mainly limited to a reduction in the percentage of participants with severe or moderate vision loss, defined as a loss of 15 and 30 or more letters of vision, respectively, at 1 and 2 years. In contrast only a small percentage of participants treated with verteporfin—PDT or intravitreal Macugen® have an improvement in visual acuity over baseline values.

Because irreversible retinal damage due to exudative AMD is the direct result of abnormal choroidal blood vessel growth beneath the retina and/or the retinal pigment epithelium (RPE), a number of angiostatic agents are now being evaluated clinically for use in treating this blinding disorder. Angiogenesis is a complex of inter-related processes with numerous potential opportunities for therapeutic intervention.

### SUMMARY OF THE INVENTION

The present invention overcomes these and other drawbacks of the prior art by providing a method for treating pathologic ocular angiogenesis, which includes posterior segment neovascularization. Pathologic ocular neovascularization is the vision-threatening pathology responsible for the two most common causes of acquired blindness in developed countries: age-related macular degeneration and proliferative diabetic retinopathy. Thus, the present invention provides a method for treating pathologic ocular angiogenesis, such as age-related macular degeneration, choroidal neovascularization, or proliferative diabetic retinopathy. The method of the invention includes administering to a patient in need thereof a combination of anecortave acetate and bevacizumab or ranibizumab.

In preferred aspects of the invention, the anecortave acetate is administered via posterior juxtascleral depot and the bevacizumab or ranibizumab is administered intravitreally. Typically, the amount of anecortave acetate administered is from 3 mg to 30 mg and the amount of bevacizumab is from 0.1 mg to 5 mg. In alternative embodiments, the amount of anecortave acetate administered is from 3 mg to 30 mg and the amount of ranibizumab administered is from 0.05 mg to 5 mg. Most preferably, the amount of anecortave acetate administered is 15 mg and the amount of bevacizumab administered is 1 mg. In another embodiment of the invention, the amount of anecortave acetate administered is 15 mg and the amount of ranibizumab administered is 0.5 mg.

In preferred embodiments of the invention, the administration of bevacizumab is repeated at intervals of no less than six weeks. In another preferred embodiment, the administration of ranibizumab is repeated at intervals of one month to three months. The administration of anecortave acetate will be repeated at intervals of no more than six months. The need for subsequent administrations of bevacizumab or ranibizumab and anecortave acetate will be determined by the skilled physician.

### DETAILED DESCRIPTION PREFERRED EMBODIMENTS

Anecortave acetate is an angiostatic agent developed by Alcon Research, Ltd. for the inhibition of ocular neovascularization. Anecortave acetate is a synthetic derivative of cortisol acetate with specific and irreversible chemical modifications made to its original structure. Removal of the 11-beta hydroxyl and the addition of a new double bond at the C9-11 position resulted in a novel angiostatic cortisene that does not exhibit the typical undesirable side effects of glucocorticoids. These modifications resulted in the elimination of glucocorticoid receptor-mediated activities typical of the original cortisol acetate molecule. Preclinical studies have demonstrated the angiostatic efficacy of anecortave acetate in a wide variety of animal models of ocular neovascularization. In addition, anecortave acetate has an excellent ocular and systemic safety profile and is successfully delivered transcerally to the back of the eye following both single and multiple periocular posterior juxtascleral administrations.

Bevacizumab binds VEGF and prevents the interaction of VEGF with its receptors (Flt-1 and KDR) on the surface of endothelial cells. The interaction of VEGF with its receptors leads to endothelial cell proliferation and new blood vessel formation in in vitro models of angiogenesis. Administration of bevacizumab to xenotransplant models of colon cancer in nude (athymic) mice caused reduction of microvascular growth and inhibition of metastatic disease progression (Presta et al. 1997).

Ranibizumab is a recombinant humanized IgG1 kappa isotype monoclonal antibody fragment of bevacizumab, having a molecular weight of approximately 48 kilodaltons, which was designed for intraocular use. It binds to and inhibits the biologic activity of human vascular endothelial growth factor A (VEGF-A). The binding of rabibizumab to VEGF-A prevents the interaction of VEGF-A with its receptors, VEGFR1 and VEGFR2, on the surface of endothelial cells, reducing endothelial cell proliferation, vascular leakage and new blood vessel formation.

In contrast to other experimental therapies for AMD, which were designed to specifically inhibit angiogenesis stimulated by vascular endothelial growth factor (VEGF) (The EyeTech Study Group 2002; Krzystolik et al. 2002), anecortave acetate inhibits blood vessel growth by inhibiting the proteases necessary for vascular endothelial cell migration (DeFaller and Clark 2000; Penn et al. 2001). Anecortave acetate is unique in that it inhibits angiogenesis subsequent to (and therefore independently of) the actual angiogenic stimulus, and it therefore has the potential to nonspecifically inhibit angiogenesis driven by the wide variety of known ocular angiogenic stimuli (Casey and Li 1997). The ability of anecortave acetate to inhibit angiogenesis independently of the initiating stimulus is supported by a large body of preclinical evidence, including multiple animal models of neovascularization (Penn et al. 2001; Clark 1997; McNatt et al. 1999; BenEzra et al. 1997).

The combination therapy of the present invention provides an agent acting directly on the actual angiogenic stimulus (e.g., bevacizumab or ranibizumab) and an agent that inhibits angiogenesis subsequent to the angiogenic stimulus (e.g., anecortave acetate), thus providing an effective means for the treatment of disorders resulting from pathologic ocular angiogenesis.

The formulations for use in the methods of the invention can be delivered by intravitreal, posterior juxtascleral, or subconjunctival injection as well as via an implanted device as further below described. All cited patents are herein incorporated by reference.

Particularly preferred implanted devices include: various solid and semi-solid drug delivery implants, including both non-erodible, non-degradable implants, such as those made using ethylene vinyl acetate, and erodible or biodegradable implants, such as those made using polyanhydrides or polylactides. Drug delivery implants, particularly ophthalmic drug delivery implants are generally characterized by at least one polymeric ingredient. In many instances, drug delivery implants contain more than one polymeric ingredient.

For example, U.S. Pat. No. 5,773,019 discloses implantable controlled release devices for delivering drugs to the eye wherein the implantable device has an inner core containing an effective amount of a low solubility drug covered by a non-bioerodible polymer coating layer that is permeable to the low solubility drug.

U.S. Pat. No. 5,378,475 discloses sustained release drug delivery devices that have an inner core or reservoir comprising a drug, a first coating layer which is essentially impermeable to the passage of the drug, and a second coating layer which is permeable to the drug. The first coating layer covers at least a portion of the inner core but at least a small portion of the inner core is not coated with the first coating layer. The second coating layer essentially completely covers the first coating layer and the uncoated portion of the inner core.

U.S. Pat. No. 4,853,224 discloses biodegradable ocular implants comprising microencapsulated drugs for implantation into the anterior and/or posterior chambers of the eye. The polymeric encapsulating agent or lipid encapsulating agent is the primary element of the capsule.

U.S. Pat. No. 5,164,188 discloses the use of biodegradable implants in the suprachoroid of an eye. The implants are generally encapsulated. The capsule, for the most part, is a polymeric encapsulating agent. Material capable of being placed in a given area of the suprachoroid without migration, “such as oxycel, gelatin, silicone, etc.” can also be used.

U.S. Pat. No. 6,120,789 discloses the use of a non-polymeric composition for in situ formation of a solid matrix in an animal, and use of the composition as a medical device or as a sustained release delivery system for a biologically-active agent, among other uses. The composition is composed of a biocompatible, non-polymeric material and a pharmaceutically acceptable, organic solvent. The non-polymeric composition is biodegradable and/or bioerodible, and substantially insoluble in aqueous or body fluids. The organic solvent solubilizes the non-polymeric material, and has a solubility in water or other aqueous media ranging from miscible to dispersible. When placed into an implant site in an animal, the non-polymeric composition eventually transforms into a solid structure. The resulting implant provides a system for delivering a pharmaceutically effective active agent to the animal. According to the '789 patent, suitable organic solvents are those that are biocompatible, pharmaceutically acceptable, and will at least partially dissolve the non-polymeric material. The organic solvent has a solubility in water ranging from miscible to dispersible. The solvent is capable of diffusing, dispersing, or leaching from the composition in situ into aqueous tissue fluid of the implant site such as blood serum, lymph, cerebral spinal fluid (CSF), saliva, and the like. According to the '789 patent, the solvent preferably has a Hildebrand (HLB) solubility ratio of from about 9-13 (cal/cm3)1/2 and it is preferred that the degree of polarity of the solvent is effective to provide at least about 5% solubility in water.

Polymeric ingredients in erodible or biodegradable implants must erode or degrade in order to be transported through ocular tissues and eliminated. Low molecular weight molecules, on the order of 4000 or less, can be transported through ocular tissues and eliminated without the need for biodegradation or erosion.

Another implantable device that can be used to deliver formulations of the present invention is the biodegradable implants described in U.S. Pat. No. 5,869,079.

It should be appreciated that anecortave acetate or its corresponding alcohol (4,9(11)-pregnadien-17α,21-diol-3,20 dione) can also be administered via a juxtascleral implant as described, e.g., in the following commonly owned patents and patent applications: U.S. Pat. Nos. 6,413,540B1; 6,416,777B1; WO/03/009784; and WO/03/009774. Juxtascleral administration via depot or by any other method provides for transcleral delivery of the drug. It can also be administered by an intravitreal injection or an implant, such as the one described in a co-pending U.S. application publication number US 2003/0176854.

In most preferred aspects of the invention, anecortave acetate will be delivered via posterior juxtascleral administration. For posterior juxtascleral delivery of anecortave acetate, the preferred device is disclosed in commonly owned U.S. Pat. No. 6,413,245 B1 (cannula).

It is contemplated that the amount of anecortave acetate administered to the patient will be from 3 mg to 30 mg. It is most preferred that 15 mg of anecortave acetate be administered to the patient via posterior juxtascleral administration. The amount of bevacizumab to be administered is preferably from 0.1 mg to 5 mg. More preferably, 1 mg of bevacizumab will be administered by intravitreal injection. The amount of ranibizumab to be administered is preferably from 0.05 mg to 5 mg. More preferably, 0.5 mg of ranibizumab will be administered by intravitreal injection.

Typically, the initial administrations of anecortave acetate and bevacizumab or ranibizumab will occur within a few days and preferably will occur on the same day. Subsequent administrations of bevacizumab will occur at six week intervals. If necessary, subsequent administrations of bevacizumab may occur one the three days prior to the day that is six weeks after the previous administration. However, it is preferable that subsequent administrations occur on or after the day that is six weeks after the previous administration. Subsequent administrations of ranibizumab will occur at intervals of one month to three months. In certain embodiments, the administration of ranibizumab will occur at intervals of one month for the first two to six months of administration, and at intervals of three months thereafter. Preferably, the administration of ranibizumab will occur at intervals of one month for the first four months, and at intervals of three months thereafter. Subsequent administrations of anecortave acetate will occur no more than six months after the previous administrations.

The preferred compositions of the present invention are intended for administration to a human patient suffering from pathologic ocular angiogenesis and/or any associated edema. Examples of diseases or disorders encompassed by pathologic ocular angiogenesis and any associated edema include, but are not limited to: age-related macular degeneration, diabetic retinopathy, chronic glaucoma, retinal detachment, sickle cell retinopathy, rubeosis iritis, uveitis, neoplasms, Fuch's heterochromic iridocyclitis, neovascular glaucoma, corneal neovascularization, neovascularization resulting from combined vitrectomy and lensectomy, retinal ischemia, choroidal vascular insufficiency, choroidal thrombosis, carotid artery ischemia, retinal artery/vein occlusion, e.g., central retinal artery occlusion and branch retinal vein occlusion, contusive ocular injury, and retinopathy of prematurity.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### EXAMPLE 1

### Initial Administrations of Bevacizumab and Anecortave Acetate

Intravitreal Bevacizumab

Intravitreal bevacizumab injections will be administered on the same day as and prior to juxtascleral anecortave acetate administration. The vials containing bevacizumab will be maintained at 4° C., and shaken well for at least one minute before using. The eye will be washed and draped in usual sterile fashion. Topical anesthesia will be given and a speculum will be placed for adequate exposure. The injection quadrant will be chosen by the treating physician and the site for injection measured at 3.0 to 4.0 mm posterior to the limbus. A 28- or 30-gauge needle will be used to administer a 50 μL injection of the drug. After injection, a paracentesis will be preformed at the treating physician's discretion and the speculum will be removed.

Juxtascleral Anecortave Acetate

Anecortave acetate will be delivered using a specially designed curved cannula, as described in U.S. Pat. No. 6,413,245 B1. The administration procedure requires surgical expertise, because the conjunctiva and TEnon's capsule must be dissected down to bare sclera and the cannula inserted along the tissue plane between Tenon's capsule and the external scleral surface to ensure that the material is in direct apposition to the sclera near the macula. When 0.5 ml of a composition containing 30 mg/ml of anecortave acetate is introduced onto the outer scleral surface through the cannula at a slow steady rate (over at least 10 seconds), the space in this tissue plane expands to accommodate the administered material. As this expansion of the posterior juxtascleral space is occurring, some residual backflow or reflux of material along the cannula track and out at the incision site can occur. Reflux of material during administration can be minimized or prevented by using a slow steady rate of administration and by application of gentle pressure with a counter pressure device (CPD) during administration of material and withdrawal of the cannula.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and structurally related may be substituted for the agents described herein to achieve similar results. All such substitutions and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

United States Patents and Patent Applications

4,853,224

5,164,188

5,378,475

5,773,019

5,869,079

6,120,789

6,413,245 B1

6,413,540 B1

6,416,777

U.S. application Ser. No. 10/385,791

Foreign Patents and Patent Applications

WO/03/009774

WO/03/009784

Other Publications
BenEzra D, Griffin B W, Naftzir G, Sharif N A and Clark A F. Topical formulations of novel angiostatic steroids inhibit rabbit corneal neovascularization. Invest. Ophthalmol. Vis. Sci. 1997; 38: 1954-62.
Bressler et al. Clinicopathologic correlation of drusen and retinal pigment epithelial abnormalities in age-related macular degeneration. Sur Ophthalm. 1988; 32(6):375-413.
Casey R, Li W W. Factors Controlling Ocular Angiogenesis. Amer. J. Ophthalmol. 1997; 124: 521-529.
Clark A F. AL-3789: a novel ophthalmic angiostatic steroid. Exp. Opin. Invest. Drugs 1997; 6: 1867-77.
DeFaller J M and Clark A F. A new pharmacological treatment for angiogenesis. In Pterygium, Taylor, HR (ED.) The Hague: Kugler Publications, 2000; 159-181.
Krzystolik M G, Afshari M A, Adamis A P, et al. Prevention of experimental choroidal neovascularization with intravitreal anti-vascular endothelial growth factor antibody fragment. Arch. Ophthalmol. 2002; 120: 338-46.
McNatt L G, Weimer L, Yanni J and Clark A F. Angiostatic activity of steroids in the chick embryo CAM and rabbit cornea models of neovascularization. J. Ocular Pharm. Therap. 1999; 15(5): 413-23.
Penn J S, Rajaratnam V S, Collier R J and Clark A F. The effect of an angiostatic steroid on neovascularization in a rat model of retinopathy of prematurity. Invest. Ophthalmol. Vis. Sci. 2001; 42: 283-90.
Presta L G, Chen H, O'Connor S J, Chisholm V, Meng Y G, Krummen L, et al. Humanization of an anti-vascular endothelial growth factor monoclonal antibody for the therapy of solid tumors and other disorders. Cancer Res 1997; 57:4593-9.
Seddon J M. Epidemiology of age-related macular degeneration. Retina, Ryan S J (ED.). St. Louis: Mosby, 2001; 1039-50.
The EyeTech Study Group. Preclinical and phase 1A clinical evaluation of an anti-VEGF pegylated aptamer (EYE001) for the treatment of exudative age-related macular degeneration. Retina 2002; 22: 143-52.
Treatment of Age-related Macular Degeneration with Photodynamic Therapy (TAP) Study Group. Photodynamic therapy of subfoveal choroidal neovascularization in age-related macular degeneration with verteporfin—TAP Report 1. Arch. Ophthalmol. 1999; 117: 1329-45.

We claim:

1. A method for treating age-related macular degeneration, comprising administering to a patient in need thereof anecortave acetate and bevacizumab.

2. The method of claim 1, wherein the anecortave acetate is administered via posterior juxtascleral depot and the bevacizumab is administered intravitreally.

3. The method of claim 1, wherein the amount of anecortave acetate administered is from 3 mg to 30 mg and the amount of bevacizumab is from 0.1 mg to 5 mg.

4. The method of claim 3, wherein the amount of anecortave acetate administered is 15 mg and the amount of bevacizumab administered is 1 mg.

5. The method of claim 1, wherein the administration of bevacizumab is repeated at six week intervals.

6. The method of claim 1, wherein the administration of anecortave acetate is repeated at six month intervals.

7. A method for treating choroidal neovascularization, comprising administering to a patient in need thereof anecortave acetate and bevacizumab.

8. The method of claim 7, wherein the anecortave acetate is administered via posterior juxtascleral depot and the bevacizumab is administered intravitreally.

9. The method of claim 7, wherein the amount of anecortave acetate administered is from 3 mg to 30 mg and the amount of bevacizumab is from 0.1 mg to 5 mg.

10. The method of claim 9, wherein the amount of anecortave acetate administered is 15 mg and the amount of bevacizumab administered is 1 mg.

11. The method of claim 7, wherein the administration of bevacizumab is repeated at six week intervals.

12. The method of claim 7, wherein the administration of anecortave acetate is repeated at six month intervals.

13. A method for treating age-related macular degeneration, comprising administering to a patient in need thereof anecortave acetate and ranibizumab.

14. The method of claim 13, wherein the anecortave acetate is administered via posterior juxtascleral depot and the ranibizumab is administered intravitreally.

15. The method of claim 13, wherein the amount of anecortave acetate administered is from 3 mg to 30 mg and the amount of ranibizumab is from 0.05 mg to 5 mg.

16. The method of claim 15, wherein the amount of anecortave acetate administered is 15 mg and the amount of ranibizumab administered is 0.5 mg.

17. The method of claim 13, wherein the administration of ranibizumab is repeated at one month intervals.

18. The method of claim 13, wherein the administration of ranibizumab is repeated at three month intervals.

19. The method of claim 13, wherein the administration of ranibizumab is repeated at one month intervals for two to six months and at four month intervals thereafter.

20. The method of claim 13, wherein the administration of anecortave acetate is repeated at six month intervals.

21. A method for treating choroidal neovascularization, comprising administering to a patient in need thereof anecortave acetate and ranibizumab.

22. The method of claim 21, wherein the anecortave acetate is administered via posterior juxtascleral depot and the ranibizumab is administered intravitreally.

23. The method of claim 21, wherein the amount of anecortave acetate administered is from 3 mg to 30 mg and the amount of ranibizumab is from 0.05 mg to 5 mg.

24. The method of claim 23, wherein the amount of anecortave acetate administered is 15 mg and the amount of ranibizumab administered is 0.5 mg.

25. The method of claim 21, wherein the administration of ranibizumab is repeated at one month intervals.

26. The method of claim 21, wherein the administration of ranibizumab is repeated at three month intervals.

27. The method of claim 21, wherein the administration of ranibizumab is repeated at one month intervals for two to six months and at three month intervals thereafter.

28. The method of claim 21, wherein the administration of anecortave acetate is repeated at six month intervals.
